(19) United States
(12) Patent Application Publication
(10) Pub. No.: US 2001/0029951 A1
(43) Pub. Date: Oct. 18, 2001

(54) Supplied air snorkeling system

(76) Inventor: Bela Miklos, San Diego, CA (US)

Correspondence Address: Todd A. Rathe, FOLEY & LARDNER, Firstar Center, 777 East Wisconsin Avenue, Milwaukee, WI, 53202-5367, US

(21) Appl. No.: 09/756,940

(22) Filed: Jan. 9, 2001

Related U.S. Application Data
(60) Provisional application No. 60/176,481, filed on Jan. 17, 2000.

(51) Int. Cl. A62B 7/04; A61M 16/00; F16K 31/26; A62B 7/00; A62B 9/00

(52) U.S. Cl. 128/205.22; 128/204.26

(57) ABSTRACT

A supplied air snorkeling system includes an air tank, at least one regulator coupled to the air tank and including a mouthpiece through which air from the tank is provided and a supplied air snorkeling vest. The vest includes a back portion, a front portion, a head opening and a tank holder. The back portion is configured to extend along a snorkeler's back when the vest is worn and includes at least one layer of floatation material. The front portion is pivotally coupled to the back portion and includes a main body having at least one layer of floatation material and shoulder extensions extending from the main body to the back portion. The main body is configured to extend laterally across the snorkeler's chest proximate to the snorkeler's clavicle and the across the snorkeler's sternum body. The head opening is formed at a juncture of the back portion and the front portion. As a result, the front portion may be pulled from behind the snorkeler's head and over the snorkeler's head as the head opening receives the snorkeler's head. The tank holder is coupled to the back portion and releasably secures the air tank to the back portion. A buoyancy compensator similar to the vest is also disclosed, except that the buoyancy compensator includes inflatable chambers in lieu of the flotation material.

### FIELD OF THE INVENTION

The present application claims priority under 35 U.S.C. §119(e) from U.S. Provisional Patent Application Serial No. 60/176,481, the full disclosure of which is hereby incorporated by reference.

The present invention relates to snorkeling equipment. More specifically, the present invention relates to a supplied air snorkeling system having a device that keeps the user floating on the surface of the water and includes an apparatus for holding an air supply thereon.

### BACKGROUND OF THE INVENTION

Conventional snorkeling equipment includes a mouthpiece connected to a rigid breathing tube, which extends out of the water and into the air while the snorkeler's face is under the surface of the water. Conventional snorkeling equipment may also include goggles and a life preserver.

Modified snorkeling equipment also has been proposed. One type of modified snorkeling equipment includes a long, flexible air hose having one opening that is secured to a float on the surface of the water and another opening that is placed in a snorkeler's mouth. Such snorkeling equipment allows the user to snorkel at greater distances from the surface of the water than conventional snorkeling equipment. It is shown in U.S. Pat. No. 5,606,967.

Another type of modified snorkeling equipment is a toy scuba apparatus, as shown in U.S. Pat. No. 2,975,439. This apparatus includes a simulated air tank with a snorkeling breathing conduit extending therethrough. The simulated air tank does not hold an air supply but merely functions as a buoyant float and a snorkeling device. A harness having straps attached thereto is laced to the flotation tank, and the straps are used to secure the flotation tank to a snorkeler's back. This apparatus further includes a mouthpiece and air conduit means secured to the flotation tank at one end and secured to a mouthpiece at the other end. This mouthpiece and air conduit function with the snorkel breathing conduit in the flotation tank as a snorkeling system.

One disadvantage of currently available snorkeling equipment is that a user is not necessarily kept on the surface of the water because a life preserver is not coupled with the breathing apparatus. In fact, some equipment even encourages the user to explore lower depths of the water. Another disadvantage with conventional snorkeling equipment is that it is difficult to learn the breathing techniques necessary to use the mouthpiece and breathing tube of the snorkeling equipment.

Thus, there is continuing need for a device to keep the user on the surface of the water and that hold a supply of air so that the user may be supplied with air without learning snorkeling breathing techniques.

### SUMMARY OF THE INVENTION

One embodiment of the invention relates to a supplied air snorkeling system which includes an air tank, at least one regulator coupled to the air tank and including a mouthpiece through which air from the tank is provided, and a supplied air snorkeling vest. The vest includes a back portion, a front portion, a head opening and a tank holder. The back portion is configured to extend along a snorkeler's back when the vest is worn and includes at least one layer of floatation material. The front portion is coupled to the back portion and is configured to extend laterally across the snorkeler's chest when the vest is worn. The front portion also includes at least one layer of floatation material. The head opening is formed at a juncture of the back portion and the front portion. The tank holder is coupled to the back portion and releasably secures the tank to the back portion.

One embodiment of the present invention relates to a supplied air snorkeling system including an air tank, at least one regulator coupled to the air tank and having a mouthpiece through which air from the tank is provided and a supplied air snorkeling vest. The vest includes a back portion, a front portion, a head opening and a tank holder. The back portion is configured to extend along a snorkeler's back when the vest is worn and includes at least one layer of floatation material. The front portion is pivotally coupled to the back portion and includes a main body having at least one layer of floatation material and shoulder extensions extending from the main body to the back portion. The main body is configured to extend laterally across the snorkeler's chest proximate to the snorkeler's clavicle and the across the snorkeler's sternum body. The head opening is formed at a juncture of the back portion and the front portion. As a result, the front portion may be pulled from behind the snorkeler's head, over the snorkeler's head as the head opening receives the snorkeler's head. The tank holder is coupled to the back portion and releasably secures the air tank to the back portion.

One embodiment of the present invention relates to a supplied air snorkeling vest which includes a back portion, a front portion, a head opening and a tank holder. The back portion is configured to extend along a snorkeler's back when the vest is worn and includes at least one layer of floatation material. The front portion is pivotally coupled to the back portion and includes a main body having at least one layer of floatation material and configured to extend laterally across the snorkeler's chest approximate to the snorkeler's clavicle and across the snorkeler's sternum body. The front portion further includes shoulder extensions extending from the main body to the back portion. The head opening is formed at a juncture of the back portion and the front portion. As a result, the front portion may be pulled from behind the snorkeler's head, over the snorkeler's head as the head opening receives the snorkeler's head. The tank holder is coupled to the back portion and releasably secures the tank to the back portion.

One embodiment of the present invention relates to a buoyancy compensator including a back portion, a front portion, a head opening, first and second shoulder strap assemblies, first and second side portions and a tank holder. The back portion is configured to extend along the diver's back when the buoyancy compensator is worn. The back portion includes at least one inflatable chamber. The front portion is pivotally coupled to the back portion and includes a main body having at least one inflatable chamber. The front portion is configured to extend laterally across the diver's chest proximate to the diver's clavicle and across the diver's sternum body. The front portion additionally includes shoulder extensions extending from the main body to the back portion. The first and second shoulder strap assemblies are coupled to the back portion. The first and second side portions extend from the back portion and are configured to wrap about the diver's torso and to be releasably coupled to the front portion. The head opening is formed at a juncture of the back portion and the front portion such that the front portion may be pulled from behind the diver's head and over the diver's head as the head opening receives the diver's head. The tank holder is coupled to the back portion and releasably secures the tank to the back portion.

### BRIEF DESCRIPTION OF THE DRAWINGS

FIG. 1 is a side elevational view of an exemplary embodiment of a supplied air snorkeling system being worn by a user snorkeling.

FIG. 2 is a perspective view of the supplied air snorkeling system of FIG. 1 in an open state, portions of the system being broken away for purposes of illustration.

FIG. 3 is a front perspective view of the supplied air snorkeling system of FIG. 2 in a partially closed state.

FIG. 4 is a front perspective view of the supplied air snorkeling system of FIG. 2 in a fully closed state.

FIG. 5 is a rear elevational view of the supplied air snorkeling system of FIG. 2.

FIG. 6 is a front perspective view of a buoyancy compensator incorporating several features of the supplied air snorkeling system of FIGS. 1 through 5, portions of the system being broken away for purposes of illustration.

### DETAILED DESCRIPTION OF THE PREFERRED EMBODIMENTS

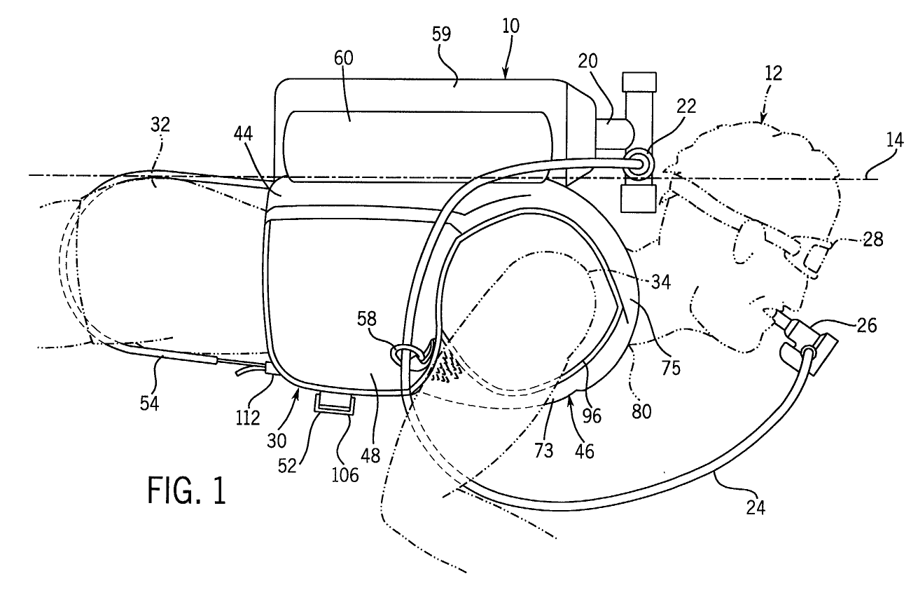

FIG. 1 is a side elevational view of supplied air snorkeling system 10 being worn by a user or snorkeler 12 during snorkeling activity in which snorkeler 12 attempts to attain a generally horizontal orientation along the water surface 14 to view underwater aquatic life and underwater structures. System 10 generally includes air tank 20, first stage regulator 22, hose 24, second stage regulator 26, optional mask 28 and supplied air snorkeling vest 30. Air tank 20, first stage regulator 22, hose 24 and second stage regulator 26 are conventionally known and provide gas or air to snorkeler 12 while snorkeler 12 is engaged in snorkeling activity along water surface 14. Examples of such first and second stage regulators are disclosed in U.S. Pat. Nos. 5,950,622; 5,503,142; 5,690,100; 5,687,712; 5,191,317; and 5,190,030 (the full disclosures of which are hereby incorporated by reference).

Mask 28 is optional and serves to protect the snorkeler's eyes from the surrounding water. In the exemplary embodiment, mask 28 is illustrated for use with nose plugs. Alternatively, mask 28 may be configured to simultaneously occlude or surround the snorkeler's nostrils. An exemplary mask or goggles is disclosed in U.S. Pat. No. 4,856,120 (the full disclosure of which is hereby incorporated by reference).

Vest 30 mounts to tank 20 and supports tank 20 along the snorkeler's back when vest 30 is worn, as shown in FIG. 1. Vest 30 further retains hose 24 and second stage regulator 26 adjacent to the body of snorkeler 12. Vest 30 includes one or more layers of flotation material, such as flotation foam, extending along both the back of snorkeler 12 and the chest of snorkeler 12. Vest 30 is specifically configured to buoyantly support the butt 32 and the shoulders 34 of snorkeler 12 in a substantially horizontal, level orientation relative to water surface 14 such that the head of snorkeler 12 may be easily lifted above water surface 14. This is facilitated by vest 30 including flotation material that extends laterally across the snorkeler's chest, at least from a point proximate the snorkeler's clavicle to a point proximate the snorkeler's xiphoid process and laterally across the sternum body of snorkeler 12. Although conventionally known, the definitions for such anatomical terms may be found in Atlas of Human Anatomy by Frank H. Netter which was published by Novartis Pharmaceuticals Corporation, 556 Morris Avenue, Summit, N.J. 07901 in 1997 (the full disclosure of which is hereby incorporated by reference). Because vest 30 employs flotation material, vest 30 cannot be inflated or deflated and substantially prohibits the user or snorkeler from submerging. As a result, vest 30 and snorkeling system 10 is extremely well suited for use by children or others who are just becoming accustomed to water, who are not proficient swimmers, who have not learned the breathing techniques required to use a mouthpiece and breathing tube of conventional snorkeling equipment or who are learning the breathing techniques associated with scuba diving equipment.

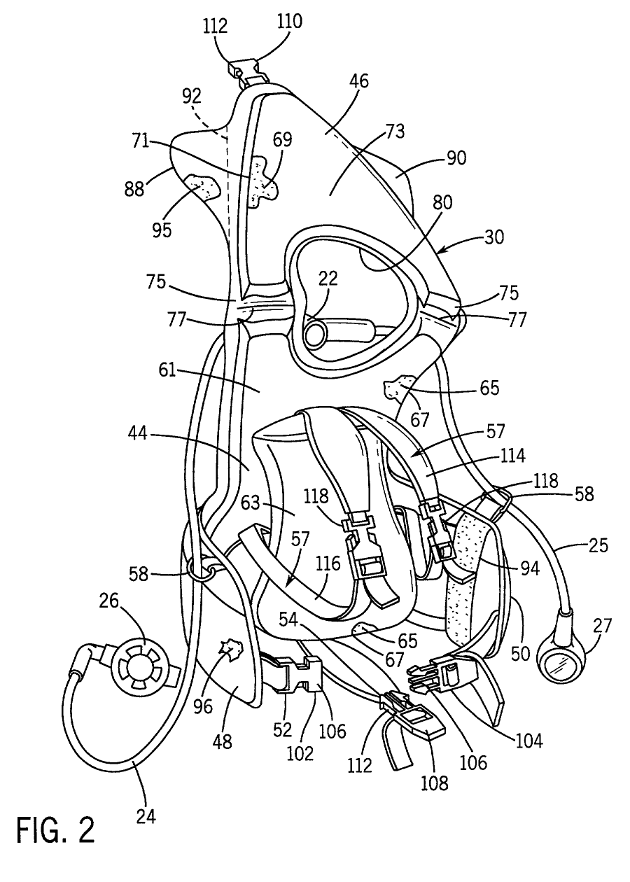

FIGS. 2-5 illustrate vest 30 in greater detail. As best shown by FIG. 2, vest 30 generally includes back portion 44, front portion 46, side portions 48, 50, belt 52, lower connection strap 54, shoulder strap mechanisms 57, hose retainers 58, tank holder 59 and back flotation assists 60. Back portion 44 comprises a buoyant panel configured to extend adjacent the snorkeler's back. Preferably, the panel forming back portion 44 is composed of one or more layers of buoyant flotation foam coated with a wear-resistant layer or disposed within a pocket of wear-resistant fabric such as nylon. In the exemplary embodiment, back portion 44 preferably includes a main panel 61 and an auxiliary panel 63. Auxiliary panel 63 comprises a panel sewn or otherwise coupled to main panel 61 at a lower end and releasably secured to main panel 61 by a hook and loop fastener system (not shown) between panels 61 and 63. Panels 61 and 63 each preferably comprise one or more layers of buoyant floatation foam 65 disposed within a pocket of wear resistant fabric 67 such as nylon. As a result, the positioning of auxiliary panel 63 relative to main panel 61 may be adjusted and repositioned depending upon the anatomy of the snorkeler wearing vest 30.

Front portion 46 comprises an elongate generally triangular-shaped tapering panel extending from back portion 44. The panel forming front portion 46 is preferably formed from one or more layers of buoyant flotation foam 69 coated with a wear-resistant material or disposed within a pocket of wear-resistant material 71 such as nylon. In the exemplary embodiment, front portion 16 is integrally formed as part of a single unitary body with back portion 44 and includes main body 73 and shoulder extensions 75. Main body 73 is configured to extend below the snorkeler's neck, laterally across the snorkeler's chest, across the sternum body and between the snorkeler's clavicle and xiphoid process. Shoulder extension 75 extends from main body 73 to main panel 61 of back portion 44. As best shown by FIG. 2, shoulder extensions 75 each include a portion devoid of floatation material so as to provide a crease, living hinge or seam 77 joining back portion 44 and front portion 46 and pivotally coupling front portion 46 to back portion 44 about an axis extending along the snorkeler's shoulders when vest 30 is being worn.

As further shown by FIG. 2, shoulder extensions 75, front portion 46 and back portion 44 form an opening 80. Because front portion 46 is pivotally coupled to back portion 44 and because opening 80 extends at a juncture front portion 46 and back portion 44, front portion 46 may be pulled from behind the snorkeler's head and over the snorkeler's head while opening 80 receives the snorkeler's head.

As further shown by FIG. 2, front portion 46 additionally includes outwardly extending wings 88, 90 extending from opposite sides of front portion 46. Wings 88, 90 are preferably formed of a thinner layer or layers 95 of buoyant floatation material such that wings 88, 90 fold or bend along crease lines 92 to conform to a snorkeler's body. Wings 88, 90 also preferably have an outer surface provided with one member of a hook and loop fastener 94 configured to releasably attach wings 88, 90 to side portions 48, 50, respectively, by means of a second portion of a hook and loop fastener mechanism.

Side portions 48, 50 extend from back portion 44 and include at least one layer 95 of buoyant material such as flotation foam. Although side portions 48, 50 preferably comprise layers of flotation foam disposed within a pocket of wear-resistant fabric such as nylon, side portions 48 and 50 may alternatively comprise layers of buoyant flotation material coated with a wear-resistant outer layer. Side portions 48, 50 cooperate with back portion 44 and front portion 46 to provide armholes 96 shown in FIG. 4. Side portions 48, 50 further wrap about the snorkeler's sides and partially across the snorkeler's front. Side portions 48, 50 are maintained about the torso of a user by means of the aforementioned hook and loop fastener arrangement 94 in which the inner surfaces of portions 48, 50 are provided with a second portion of a hook and loop fastener 94.

Belt 52 and strap 54 further assist in securing and maintaining system 10 upon a user. Belt 52 includes opposing end portions 102, 104 which extend from side portions 48, 50, respectively. End portions 102 and 104 are preferably releasably connectable to one another by fasteners 106 such as a side release buckle. Connection strap 54 extends from back portion 44 between a snorkeler's legs and has an end 108 releasably connectable to a lower terminal end 110 of front portion 46. End 108 is releasably connectable to end 110 by means of fasteners such as a side-release buckle 112.

Shoulder strap assemblies 57 each generally include a padded upper shoulder member 114 and a lower member 116 releasably connected to upper member 114 by a fastener 118 such as a side release buckle. Upper shoulder member 114 has a first end affixed, preferably by stitching, to back portion 44 and a second end releasably secured to lower member 116 by fastener 118. Lower member 116 is secured to back portion 44 below upper portion 114. Shoulder strap assemblies 57 enable device 110 to be placed and held on a user as front portion 46 is pulled over the snorkeler's head and secured to strap 54 by means of fastener 112 comprising a side release buckle, and as side portions 48, 50 are wrapped about the snorkeler's torso and secured to one another by means of fastener 106 preferably comprising a side release buckle.

Hose retainers 58 generally comprising a sleeve or clip coupled to vest 30 while being appropriately configured and located to retain the hose 24, extending between the first stage regulator and the second stage regulator, and hose 25, extending between regulator 22 and pressure gauge 27, adjacent to vest 30. In particular applications, hose retainers 58 may be appropriately configured so as to prevent the separation of the air supply system from vest 30 by using a sleeve sized to receive the hose 24 but having an opening small enough to prevent the passage of the second stage regulator 26 therethrough, whereby the second stage regulator 26 cannot be removed from the hose 24 without tools. Where a clip is used to retain the hose adjacent to vest 30, the clip may also be appropriately configured to prevent separation of the hose 24 itself from vest 30 unless a tool is used or a correct combination is supplied to a locking mechanism provided as part of the clip. Such safety mechanisms may be optionally provided where it is desirable to prevent the air supply from being separated from vest 30 such as when system 10 is being worn by children or inexperienced swimmers.

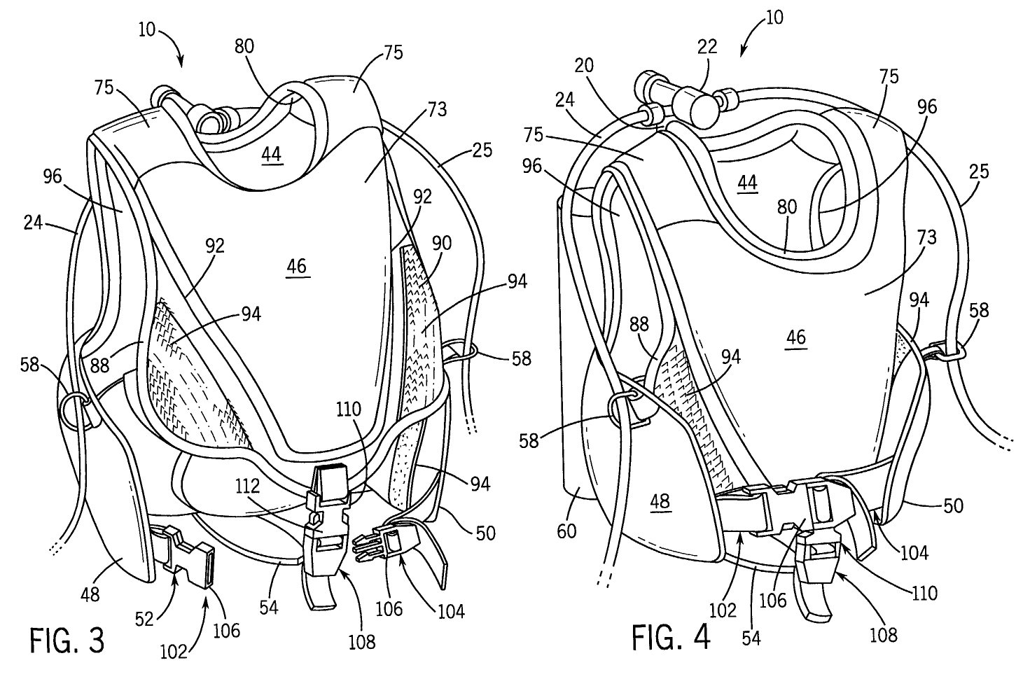

FIGS. 3 and 4 illustrate the positioning of vest 30 upon a user or snorkeler. In particular, system 10 is initially positioned such that the snorkeler's arms extend between upper shoulder member 114 and lower shoulder member 116 of shoulder strap assemblies 57 to position back portion 44 adjacent the snorkeler's back with side portions 48, 50 extending along the snorkeler's sides. Front portion 46 is then pivoted about crease 77 towards the snorkeler's front so as to insert the snorkeler's head through head opening 80. Side portions 48 and 50 are then secured to front portion 46 by means of the hook and loop fastener mechanisms 94. Hook and loop fastener mechanisms 94 retain side portions 48, 50 against front portion 46 as fasteners 106 and 112 are manipulated. Fasteners 106 are used to connect side portions 48, 50 and ends 102, 104 of belt 52. Strap 54 is also coupled to a lower terminal end of front portion 46 by means of fastener 112.

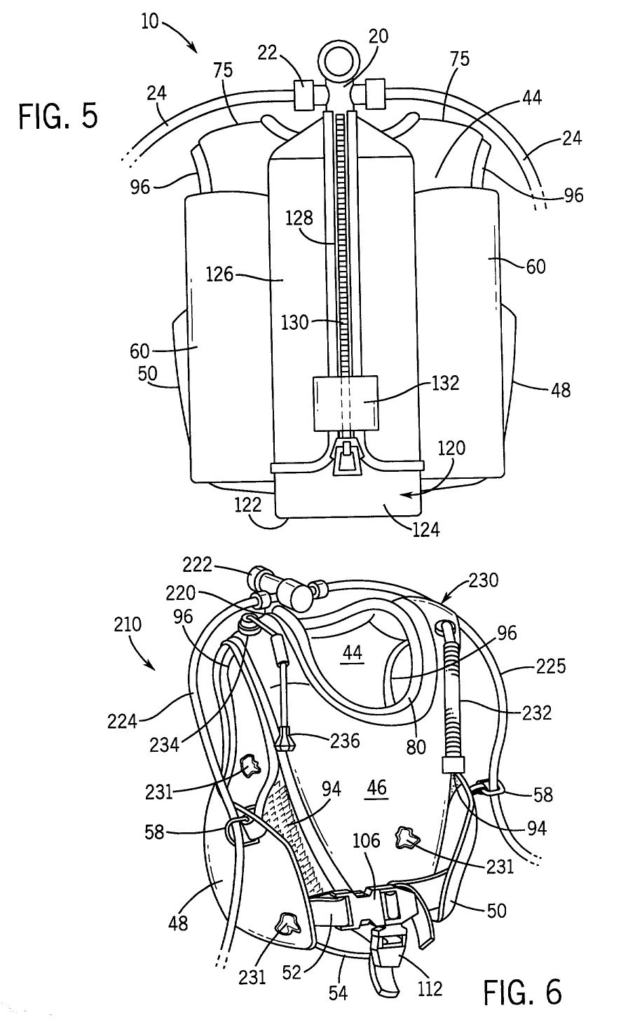

FIG. 5 illustrates tank holder 59 and back flotation assists 60 in greater detail. Tank holder 59 is coupled to main panel 61 of back portion 44 and is configured to releasably retain air tank 20 along back portion 46 and along the snorkeler's back. As shown by FIG. 5, tank holder 59 preferably comprises a cup-shaped fabric pouch 120 having a bottom 122 and a side wall 124 having an elongate slit (not shown), where portions of the side wall on opposite sides of the slit may be secured to one another about a lower end of air tank 20 by an overlapping flap having a hook and loop fastener arrangement (not shown). Tank holder 59 further includes a fabric cover 126 which is sewn to back portion 44 and which includes an elongate slit 128 that enables the cover to be separated to receive air tank 20. Once air tank 20 has been lowered into pouch 120 and has been surrounded by cover 126, opposite sides of cover 126 are further secured about air tank 20 by means of zipper 130 and securement flap 132 which is sewn to one side of cover 126 and which is releasably secured to the other side of cover 126 by a hook and loop fastener (not shown). Although less desirable, air tank 20 may be releasably mounted to back portion 44 of vest 30 by any of a variety of alternative tank holders or tank holder mechanisms such as straps, clamps, mounting braces or brackets and the like.

Back flotation assists 60 comprise generally elongate tubes or cylinders of flotation material coated with a wear resistant material or disposed within a pocket of wear resistant material such as nylon that is itself sewn, bonded or otherwise affixed to back portion 44 on opposite sides of tank holder 59 and air tank 20. Back flotation assists 60 provide additional buoyancy to buoyantly support the snorkeler along water surface 14 as shown in FIG. 1. Back flotation assists 60 preferably extend along a substantial portion of back portion 44 on opposite sides of air tank 20. Although preferably comprising elongate cylindrical members, back flotation assists 60 may have various other sizes, shapes and configurations. Although less desirable, back flotation assists 60 may be omitted depending upon the buoyancy requirements of system 10.

FIG. 6 is a front perspective view of diving system 210 including air tank 220, first stage regulator 222, hose 224 secured to a second stage regulator (not shown) and hose 225 secured to a pressure gauge (not shown). Diving system 210 additionally includes buoyancy compensator 230. Buoyancy compensator 230 is substantially identical to vest 30 except than buoyancy compensator 230 replaces flotation material 65, 69 and 96 in back portion 44, front portion 46 and side portions 48, 50, respectively, with air-tight chambers 231 configured for being inflated and deflated to adjust the buoyancy of buoyancy compensator 230. To this end, buoyancy compensator 230 additionally includes an inflation hose 232 pneumatically coupled to the chambers 231 and configured to be pneumatically coupled to hose 224 of the second stage regulator for powered inflation or to a mouthpiece for oral inflation of the interior chamber 231. Buoyancy compensator 230 further includes a conventionally known air release valve 234 which is openable and closeable via a pull cord 236 whereby air may be released from the internal chambers 231 to deflate buoyancy compensator 230 and to reduce the buoyancy of buoyancy compensator 230.

Buoyancy compensator 230 is generally similar to vest 30 in all other respects. Accordingly, those components of buoyancy compensator 230 which are similar to the corresponding components of vest 30 are numbered similarly. Like vest 30, buoyancy compensator 230 is easily and reliably positioned upon the diver. In particular, the diver simply inserts his or her arms through shoulder strap mechanisms 57, pivots front portion 46 forwardly as opening 80 receives the diver's head, releasably secures wings 88, 92, side portions 48, 50 and buckles fasteners 106 and 112 to secure belt 52 and lower connection strap 54. As a result, buoyancy compensator 230 is comfortable and enables the diver to easily dress and undress.

Although the present invention has been described with reference to preferred embodiments, workers skilled in the art will recognize that changes may be made in form and detail without departing from the spirit and scope of the invention. For example, although different preferred embodiments may have been described as including one or more features providing one or more benefits, it is contemplated that the described features may be interchanged with one another or alternatively be combined with one another in the described preferred embodiments or in other alternative embodiments. Because the technology of the present invention is relatively complex, not all changes in the technology are foreseeable. The present invention described with reference to the preferred embodiments and set forth in the following claims is manifestly intended to be as broad as possible. For example, unless specifically otherwise noted, the claims reciting a single particular element also encompass a plurality of such particular elements.

What is claimed is:

1. A supplied air snorkeling system comprising:
an air tank;
at least one regulator coupled to the air tank and including a mouthpiece through which air from the tank is provided; and
a supplied air snorkeling vest, the vest including:
a back portion configured to extend along a snorkeler's back when the vest is worn, the back portion including at least one layer of flotation material;
a front portion coupled to the back portion and configured to extend laterally across the snorkeler's chest when the vest is worn, the front portion including at least one layer of flotation material;
a head opening formed at a juncture of the back portion and the front portion; and
a tank holder coupled to the back portion and releasably securing the tank to the back portion.

2. The system of claim 1, wherein the vest includes first and second side portions extending from the back portion and configured to wrap about a snorkeler's torso.

3. The system of claim 2, wherein the first and second side portions are releasably coupled to the front portion.

4. The system of claim 3, wherein the first and second side portions are releasably coupled to the front portion by at least one hook and loop fastener mechanism.

5. The system of claim 3, wherein the front portion includes first and second wings releasably coupled to the first and second side portions.

6. The system of claim 1 including a waist belt coupled to the back portion.

7. The system of claim 1 including an intermediate strap coupled to the back portion and configured to extend between the snorkeler's legs prior to being coupled to the front portion.

8. The system of claim 1, wherein the vest includes a handle mechanism coupled to the back portion.

9. The system of claim 1, wherein the handle mechanism extends along a longitudinal center line of the back portion.

10. The system of claim 1, wherein the handle mechanism includes:
first and second strap portions coupled to the vest and extending on a first side of the tank holder; and
third and fourth strap portions coupled to the vest extending on a second opposite side of the tank holder, wherein the first and second strap portions and the third and fourth strap portions converge into a single handle along a longitudinal center line of the back portion.

11. The system of claim 1 including first and second shoulder strap assemblies coupled to the back portion.

12. The system of claim 1, wherein the vest is configured such that the front portion is pivotally coupled to the back portion proximate the snorkeler's shoulders when the vest is worn.

13. The system of claim 1, wherein the vest is configured such that the at least one layer of flotation material of the back portion and the at least one layer of flotation material of the front portion, collectively, extend substantially from the snorkeler's back to the snorkeler's front across the snorkeler's shoulders.

14. The system of claim 1, wherein the vest is configured such that the front portion and the back portion are sized and each provided with a volume of flotation material so as to suspend the snorkeler's shoulders and butt in a substantially horizontal plane during snorkeling.

15. The system of claim 1, wherein the vest is configured such that the front portion extends laterally across the snorkeler's chest adjacent to the snorkeler's clavicle.

16. The system of claim 1, wherein the vest is configured such that the front portion extends from a location proximate the snorkeler's clavicle laterally across the snorkeler's sternum body when the vest is worn by the user.

17. The system of claim 1 including at least one hose retainer coupled to the vest.

18. The system of claim 1, wherein the front portion includes a main body and shoulder extensions extending from the main body to the back portion.

19. A supplied air snorkeling system comprising:
an air tank;
at least one regulator coupled to the air tank and including a mouthpiece through which air from the tank is provided; and
a supplied air snorkeling vest, the vest including:
a back portion configured to extend along a snorkeler's back when the vest is worn, the back portion including at least one layer of flotation material;
a front portion pivotally coupled to the back portion and including a main body having at least one layer of flotation material and configured to extend laterally across the snorkeler's chest proximate to the snorkeler's clavicle and across the snorkeler's sternum body and shoulder extensions extending from the main body to the back portion;
a head opening formed at a juncture of the back portion and the front portion, wherein the front portion may be pulled from behind the snorkeler's head over the snorkeler's head as the head opening receives the snorkeler's head; and
a tank holder coupled to the back portion and releasably securing the tank to the back portion.

20. The system of claim 19 including first and second shoulder strap assemblies coupled to the back portion.

21. The system of claim 19, wherein the vest includes first and second side portions extending from the back portion and configured to wrap about the snorkeler's torso and to be releasably coupled to the front portion.

22. The system of claim 19 including a waist belt coupled to the back portion.

23. The system of claim 1, wherein the front portion and the back portion are sized and are each provided with a volume of flotation material so as to suspend the snorkeler's shoulders and butt in a substantially level horizontal plane during snorkeling.

24. A supplied air snorkeling vest comprising:
a back portion configured to extend along a snorkeler's back when the vest is worn, the back portion including at least one layer of flotation material;
a front portion pivotally coupled to the back portion and including a main body having at least one layer of flotation material and configured to extend laterally across the snorkeler's chest proximate to the snorkeler's clavicle and across the snorkeler's sternum body, and shoulder extensions extending from the main body to the back portion;
a head opening formed at a juncture of the back portion and the front portion, wherein the front portion may be pulled from behind the snorkeler's head and over the snorkeler's head as the head opening receives the snorkeler's head; and
a tank holder coupled to the back portion and releasably securing the tank to the back portion.

25. The vest portion of claim 24 including first and second shoulder strap assemblies coupled to the back portion.

26. The vest portion of claim 24 including first and second side portions extending from the back portion and configured to wrap about the snorkeler's torso and to be releasably coupled to the front portion.

27. A buoyancy compensator comprising:
a back portion configured to extend along a diver's back when the buoyancy compensator is worn, the back portion including at least one inflatable chamber;
a front portion pivotally coupled to the back portion and including a main body having an inflatable chamber and configured to extend laterally across a diver's chest proximate to the diver's clavicle and across the diver's sternum body, and shoulder extensions extending from the main body to the back portion;
first and second shoulder strap assemblies coupled to the back portion;
first and second side portions extending from the back portion and configured to wrap about the diver's torso and to be releasably coupled to the front portion;
a head opening formed at a juncture of the back portion and the front portion, wherein the front portion may be pulled from behind the diver's head and over the diver's head as the head opening receives the diver's head; and
a tank holder coupled to the back portion and releasably securing the tank to the back portion.
